(19) United States
(12) Patent Application Publication
(10) Pub. No.: US 2011/0282334 A1
(43) Pub. Date: Nov. 17, 2011

(54) DEVICE AND METHOD FOR FISTULA TREATMENT

(75) Inventor: Endrik Groenhoff, Bonn (DE)

(73) Assignee: CeramOptec Industries Inc.

(21) Appl. No.: 13/105,155

(22) Filed: May 11, 2011

Related U.S. Application Data
(60) Provisional application No. 61/389,998, filed on Oct. 5, 2010.
(60) Provisional application No. 61/333,359, filed on May 11, 2010.

(51) Int. Cl. A61B 18/08

(52) U.S. Cl. 606/15

(57) ABSTRACT

A device and method for fistula treatment are disclosed comprising a laser source, a fiber optics system and an online monitoring system. An optical fiber, radiating from its distal end in an essentially radial pattern, is inserted utilizing suitable tools for imaging, placement and insertion. Radiation is delivered until shrinkage and closure are observed and fiber optic device is removed after a few minutes. Preferred wavelengths are 980±30 nm, 1320±50 nm, 1470±60 nm and 2000±50 nm applied alone or in combination. In another embodiment, the disclosed procedure is used as a complement of conventional techniques such as fistula plugs or placement of mucosa flaps to enhance results. In another preferred embodiment, the inner layer of the tract is saturated with light sensitive substances, such as photosensitizers. Thus, homogeneous irradiation of the surface using a suitable light distributor and light/laser source causes a depth limited necrosis of the relevant tissue. Present method and device can be used successfully to treat high fistulas with less pain than fistulotomy and without risk of bowel incontinence. Procedure requires short hospitalization stays.

Domestic Priority under 35 USC 119(e).

This application claims the benefit and priority of U.S. Provisional Application Serial No. 61/389,998 filed Oct 5, 2010, entitled “Improved Device and Method for Fistula Treatment” by Endrik Groenhoff, and of U.S. Provisional Application Serial No. 61/333,359 filed May 11, 2010, entitled “Improved Device and Method for Fistula Treatment” by Endrik Groenhoff, each of which is hereby expressly incorporated by reference in its entirety as part of the present disclosure.

### BACKGROUND OF THE INVENTION

1. Field of the Invention

The present invention relates to treatment of fistulas and in particular, to safe and effective minimally invasive elimination of fistula tracts and improved healing process.

2. Information Disclosure Statement

A fistula is an abnormal channel from a hollow body cavity to the surface (for example, from the rectum to the skin) or from one cavity to another (for example, from the vagina to the bladder). A fistula may be congenital (bladder to navel), the result of a penetrating wound (skin to lung), or formed from an ulcer or an abscess (appendix abscess to vagina, or tooth socket to sinus). The repeated filling of an abscess or a wound by the fluid contents of some body cavity prevents healing and encourages the formation of a fistula.

Certain illnesses can cause fistulas to develop. Crohn's disease is a type of inflammatory bowel disease. It affects the gastrointestinal tract. Crohn's disease is a chronic disease that can cause inflammation anywhere along the digestive tract from the mouth to the anus. Fistulas are common in Crohn's disease.

A Pilonidal sinus is a blind-end tract lined with granulation tissue, which leads to a cystic cavity with usually dead or ingrown hair in it. Excessive sitting is thought to predispose people to the condition because they increase pressure on the coccyx region. The most common situation is in the postnatal region. The pilonidal sinus may communicate with the anal canal forming a Pilonidal fistula in ano. The only therapy promising success is complete excision of the abscess and its tracts with large margins. Methylene blue is used to color all tracts linked to the main one if existent. The conventional surgery consists in excising tissue upon the periosteum of the tail bone followed by abrasion to minimize reoccurrence, therefore removing a large volume of tissue, in the order of several hundred grams. In case of an open wound occurring, healing therapy takes several months. Closing the wound leads to a higher reoccurrence rate of up to 40% after 12 months. Additionally, wound covering flap techniques like the Limberg-Flap were developed to prevent bigger incisions, thus reducing reoccurrence rates. However these elaborate techniques are complex and carried out in the disadvantageous mid line region.

Anal fistula is a common gastro intestinal disease. It begins with inflammation of the mucous lining of the rectum. The area becomes an abscess as it is constantly reinfected by feces;

eventually a fistula breaks through to the skin near the anus. It is an inflammatory tract between the anal canal and skin, that is, an abnormal connection between the epithelial surface of the anal canal and the perianal skin. Anal fistulas originate from the anal glands, which are located between the two layers of the anal sphincter and which drain into the anal canal. If the outlet of these glands becomes blocked, an abscess can form which can eventually point to the skin surface. The tract formed by this process is the fistula. Abscesses can recur if the fistula seals over, allowing the accumulation of pus. It then points to the surface again, and the process repeats.

Anal abscesses and anal fistulas are different phases of the same disease whereas the abscess is the acute symptom and the fistula is the chronic successor. Before any performing any treatment of the fistula, the abscess must be addressed by opening and puncturing the cavity for relief. In the same operation phase or later, a thread-drainage in a loop which is slowly tightened over a period of days or weeks can be placed to act as a wick, guiding pus and other liquids out of the channel. Doing so, the inflammation can stabilize and the tract has time to form epithelial tissue which might close secondary fistula tracts.

Anal fistulas are irritating because of the pus-drain and in some cases formed stools that pass through the fistula from the anal canal; additionally, recurrent abscesses may lead to significant short term morbidity from pain, and create a focus for systemic spread of infection. Based on the relationship between fistulas and sphincter muscles, they may be classified as: intersphincteric, via internal sphincter to the intersphincteric space and then to the perineum; trans-sphincteric, via internal and external sphincters into the ischiorectal fossa and then to the perineum; or suprasphincteric, via intersphincteric space superiorly to above puborectalis muscle into ischiorectal fossa and then to perineum.

Several approaches have been proposed for dealing with fistulas and in particular with anal fistulas.

One option is to insert a drainage seton. The seton is a foreign material placed through the fistula tract and left in place for a long period of time to prevent abscess formation. The seton is used to strangulate the intervening tissues so that the sphincters are slowly transected. It is tightened at regular intervals until it eventually cuts through the sphincter. This is considered a safe option, although it does not definitively cure the fistula since it does not close the fistula tract. In some cases, repeated tightening of the seton is necessary every other day. Patients have to deal with persistent drainage of pus. Furthermore, the rate of fecal incontinence following this procedure has been reported to be about 60%. Seton management has thus been used as a bridge to more definitive treatment.

A surgical option is the conventional fistulotomy, which involves an operation to cut the fistula open. A narrow probe is passed into the fistula tract with the patient under anesthesia and the tissue overlying the probe is surgically divided or cut. This can be performed by excision of the tract and surrounding tissue, simple division of the tract, or gradual division. The roof of the fistula is reduplicated/divided and a wide wound groove originates. Additionally the edges are chamfered to create a shallow wound ground. To reduce reoccurrence probability, the fistula bottom can be excised completely to remove all inflammatory tissue regions. Once the fistula has been laid open, it is packed on a daily basis for a short period of time to ensure that the wound heals from the inside out. Another surgical approach is to peel out the part of the fistula tract that crosses the muscle parts without cutting healthy fractures. Next, the clean wound is sewn and covered by means of a mucosa lobe positioned above it. If the fistula is in a high position and it passes through a significant portion of the sphincter muscle, a cutting seton may be used. This involves inserting a thin tube through the fistula tract and tying the ends together outside of the body. The scion is frequently tightened over time, gradually cutting through the sphincter muscle and healing as it goes. Once the fistula tract is in a low enough position it may be laid open to speed up the process, or the seton can remain in place until the fistula is completely cured. This option minimizes scarring but can cause incontinence in some cases. A seton may also be used to keep the tract open in order to drain out the pus and all inflammatory liquids. This may take several days or even weeks. Then, the fistula tract can be removed surgically. As a consequence, surrounding tissue must be cut out with a safety margin. These surgical approaches inevitably present problems involving wound healing, impaired control, and patient discomfort.

Another surgical procedure involves creating a small endorectal flap of tissue on the inside of the rectum and pulling it down over the inside opening of the fistula tract. The flap is lifted to expose the fistula, which is then cleaned and the internal opening is sewn shut. After cutting the end of the flap on which the internal opening was, the flap is pulled down over the sewn internal opening and sutured in place. Since these flaps have to be sutured in place, sometimes there are problems with flap retraction, thus leading to a significant recurrence rate. It also involves splitting muscle tissue, a surgical procedure with inherent discomfort and potential complications.

Another approach consists in fibrin glue injection, sometimes made from the patient's own serum. It involves injecting the fistula with biodegradable glue, which should close it from the inside out, and letting it heal naturally. However, since a fluid substance is used, it tends to run out with time. As a consequence, high failure rates have been reported.

A newer alternative to block the fistula tract is using a fistula plug, which involves plugging the tract at the primary inside opening that feeds the fistula with a plug made of biological material such as porcine small intestine submucosa. The plug is fixed from the inside of the anus with suture, letting the fistula heal from the inside out. Material is remodeled into the patient's own tissue over the course of the following weeks or months. Success rate is greater than with other prior art approaches but there are still an important number of unsuccessful outcomes. Failure results include abscess formation and plug extrusion. Furthermore, using nonhuman tissue grafts represent a potential risk of infection and may even cause rejection.

Previously mentioned treatments present various disadvantages, namely incontinence problems and other discomforts, prolonged hospital stays, recurrence, risk of infection or rejection. Additionally, treatment sometimes lasts for weeks and even months and success rates has been far from acceptable.

A minimally invasive procedure has been proposed for fistula treatment using a 532 nm from frequency-doubled Nd:YAG (KTP) laser source together with fibrin glue injected into the fistula. KTP laser radiation is emitted at 20 W continuously for 20-30 seconds, delivering a total energy of 400-600 J pursuing solidification of fibrin glue. This laser fibrin glue treatment is meant to occlude the internal fistula opening, destroy chronic inflammatory tissues lining its tract, prevent healing and then block the main tract along with any secondary tracts which might not have been identified. This treatment has some advantageous implications and gains in terms of avoiding surgery: short hospital stay, expeditious return to daily activities and health services financial gains. However, the laser fibrin glue treatment causes some pain and discharge, and patients treated have had some degree of post laser pain at defecation lasting from 1 to 4 weeks after treatment.

There is therefore a need for an effective and more reliable means for eliminating fistulas, particularly anal fistulas in a minimal invasive and safe way providing a high success rate at minimum discomfort for the patient that improves on the state of the art. Present invention addresses this need.

### OBJECTIVES AND BRIEF SUMMARY OF THE INVENTION

It is an objective of the present invention to provide a device and method for improved minimally invasive treatment of fistulas such as anal fistulas.

It is another objective of the present invention to use laser energy and conveying means to effectively treat fistulas.

It is a further objective of present invention to use appropriate imaging systems to guide treatment of fistulas.

It is yet another objective to use on-line monitoring systems to achieve accurate energy dosage for assuring a safe treatment by avoiding unnecessary deep necrosis.

Briefly stated, a device and method for fistula treatment are disclosed comprising a laser source, a fiber optics system and an online monitoring system. An optical fiber, radiating from its distal end in an essentially radial pattern, is inserted utilizing suitable tools for imaging, placement and insertion. Radiation is delivered at suitable power levels until shrinkage and closure are observed and fiber optic device is removed alter a few minutes. Preferred wavelengths such as 980±30 nm, 1320±50 nm, 1470±60 nm and 2000±60 nm are applied alone or in combination. Wavelengths are chosen depending on capacity to shrink the tracts and remove or destroy the inflamed epithelial layer, to photocoagulate blood, to limit light entrance depth, or based on their antibacterial effect property to eliminate possibility of infection. In another embodiment, the disclosed procedure is used as a complement of conventional techniques such as fistula plugs or placement of mucosa flaps to enhance results. In another preferred embodiment, the inner layer of the tract is saturated with light sensitive substances, such as photosensitizers. Thus, homogeneous irradiation of the surface using a suitable light distributor and light/laser source causes a depth limited necrosis of the relevant tissue. Present method and device can be especially used successfully to treat high fistulas with less pain than fistulotomy and without risk of bowel incontinence. Procedure requires short hospitalization stays.

The above and other objects, features and advantages of the present invention will become apparent from the following description read in conjunction with the accompanying drawings (in which like reference numbers in different drawings designate the same elements).

### BRIEF DESCRIPTION OF FIGURES

FIG. 1 depicts a diagram of a preferred embodiment of present invention showing comprising parts.

FIG. 2a shows a diagram of a close-up view of a preferred embodiment of present invention in which a substance is injected through a hollow optical fiber.

FIG. 2b shows a diagram of a close-up view of a preferred embodiment of present invention in which a substance is injected through a hollow cylinder surrounded by optical fibers.

### DETAILED DESCRIPTION OF PREFERRED EMBODIMENTS

As previously mentioned, numerous approaches have been proposed for treating fistulas. Disadvantages of such approaches include incontinence problems, risk of infection, and unsatisfactory success rates. The present invention addresses prior art drawbacks by assuring safe, reliable shrinkage and closure of fistulas such as anal fistulas by applying laser energy of appropriate wavelength. Treatment options may be applied alone or in combination or as a compliment of formerly described prior art approaches.

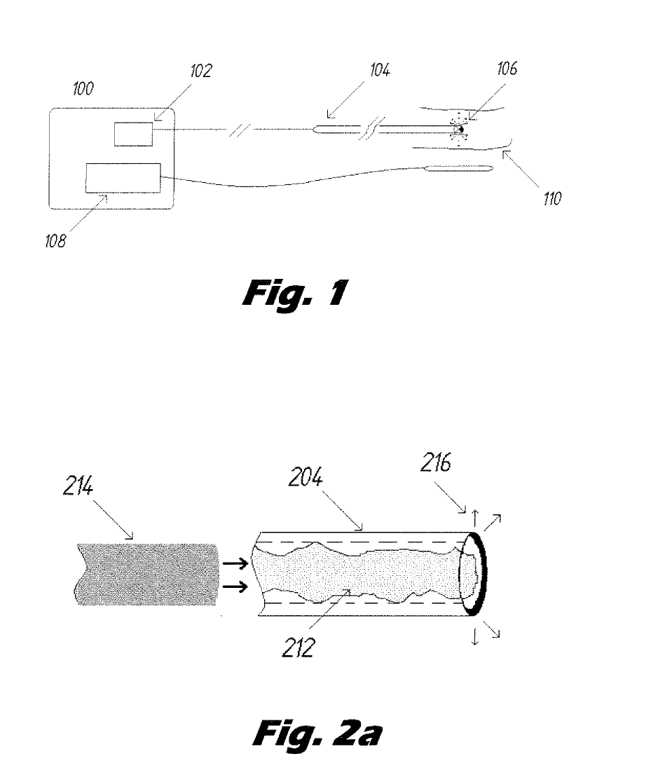

In preferred embodiments, devices for treating fistulas comprises at least one radiation source capable of producing radiation at a preselected wavelength; at least one optical waveguide, having a proximal end which is optically coupled to the radiation source and a distal end from which radiation energy is transmitted to the fistula; and a handpiece coupled to the waveguide. FIG. 1 depicts a diagram of a preferred embodiment of present invention. A laser based system 100 comprising laser source 102 emits a suitable wavelength through fiber optics device 104 radiating from its distal end 106 in an essentially radial pattern. Fiber 104 is inserted into fistula tract 110 utilizing suitable tools for imaging, placement and insertion to investigate and adjust necessary laser energy deposition. Imaging system 108 may be incorporated into device or a standalone imaging device may be employed. Imaging technology means may include but is not limited to ultrasound devices, camera vision devices, magnetic resonance tomography sets, or computed tomography sets. This allows for an on-line monitoring of the shrinking effect and for applying proper energy dosage into the fistula to prevent under or overtreatment consequences, such as causing unnecessary deep necrosis.

In a preferred embodiment, a radiation wavelength of about 1470±60 nm is used. This wavelength is advantageous for optimal shrinkage and closure effects. Radiation is preferably delivered at power levels of approximately 5-15 W. After only a few minutes, once shrinkage and closure are observed, fiber optic device 104 is removed. In another embodiment, laser source 102 emits at a wavelength of about 980±30 nm. This wavelength is ideal for achieving shrinkage of the tracts and removal of the inflamed epithelial layer. In another embodiment a wavelength of around 2 000±60 nm is applied, which limits depth of penetration, assuring non-target tissue is not affected. The wavelengths chosen in mentioned embodiments destroy bacteria in contact with or near spot of irradiation. Thus, an additional advantage of present invention is its anti-microbial effect which minimizes risk of infection. Other embodiments include but are not limited to two or more of these or other laser wavelengths applied in adequate proportion to achieve a combination of desired effects.

In another preferred embodiment, device and method of present invention are used in combination with conventional techniques mentioned in prior art to enhance results, improve successful outcome rates and to diminish reported disadvantages. For example, procedure can be an alternative to splitting the muscle layers. After removal of the abscesses, the tract can be brushed out by means of standard brushes to clean the inner fistula tract and also to intentionally create some bleeding, which complements the cleaning process. Additionally, a special endoscope with optics can be used to flood the tract with water or saline solution to improve sight and also to check for fistula branches. Commercially available endoscopes are suitable for this procedure. The remaining tracts are completely radiated from beginning to end for closure. In another embodiment, a camera is attached to the fiber. This is advantageous for cases in which an endoscope is too rugged or too big in diameter. When combined with a flap technique, a fibrin glue can be applied inside the channel. The surrounding tissue is contracted and wound healing is accelerated. In other examples, laser radiation is applied together with fistula plugs or placement of mucosa flaps. This way, fistula heals faster, there is less probability of recurrence and patient is dismissed in a considerably shorter time period.

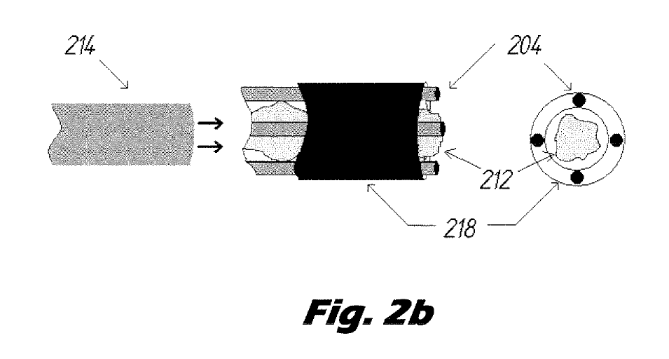

FIG. 2a shows another preferred embodiment in which fiber optic 204 is hollow and through its center, a liquid, or viscous substance 212 is driven forward on to the fistula space using a pushing device 214 that acts as a piston. Simultaneously or after injecting, irradiation 216 of fistula takes place. This way any space where the shrinkage fails to be completely close the channel will be filled up after irradiation. Substance 212 can be injected before or during laser treatment. Substance that can be injected includes but is not limited to saline solution, collagen or other suitable filler pastes. Additionally, antibacterial or healing stimulation substances can be added to the filler paste, such as antibiotics or Vascular Endothelial Growth Factor (VEGF). Another variant of present embodiment, shown in FIG. 2b, comprises several optical fibers 204 arranged circumferentially around a hollow tube 218, inside which liquid or viscous substance 212 is introduced.

In another preferred embodiment, the inner layer of the fistula tract is saturated with a photosensitizer. Photosensitizer can be introduced using the embodiment described in FIG. 2 or in parallel to a radial fiber, for example, using a micro introduction catheter. Alternatively, photosensitizer is introduced systemically. Next, after an adequate time period, using an appropriate light source for the selected photosensitizer, a suitable light distribution means is used to achieve homogeneous irradiation of the surface to be treated causing a depth limited necrosis and apoptosis of the problem tissue. When the photosensitizer absorbs emitted wavelength, a photochemical reaction causes the desired biological effect, i.e. tract is completely closed in a short period of time. As a consequence, fistula is safely treated with low risk of recurrence and with short hospital stay for the patient.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A minimally invasive method of treatment of fistulas comprising the steps of, introducing an optical fiber into a fistula ‘compartment’, and irradiating under preselected parameters to cause fistula to cure from inside out.

2. The minimally invasive method of treatment of fistulas according to claim 1, wherein said irradiating step initiates curing/disappearance of the fistula to create a field around said treatment site which is less likely to risks of infection.

3. The minimally invasive method according to claim 1, further comprising the step of introducing a substance into said fistula, prior to said irradiating step, then irradiating, establishing and maintaining a microbe free environment in the vicinity of said treatment site.

4. The minimally invasive method according to claim 3, wherein said substance is selected from a group consisting of a photosensitizer, a filler paste, collagen, saline solution, an antibacterial substance and a healing stimulation substance.

5. The minimally invasive method according to claim 1, further comprising online control via imaging technology means selected from the group of ultrasound devices, camera vision devices, magnetic resonance tomography sets and computed tomography sets to investigate and adjust necessary laser energy deposition.

6. The minimally invasive method of treatment of fistula according to claim 1 wherein said irradiating step is carried out by means of laser energy at a wavelength selected from the group of about 980±30 nm, about 1320±50 nm, 1470±60 nm and about 2000±50 nm and combinations of these.

7. A device for treating fistulas wherein an optical waveguide with a special distal end is used to irradiate preselected sites to cause shrinkage and drying up of the fistula.

8. The device for treating fistulas according to claim 7, comprising
at least one radiation source;
at least one optical waveguide, having a proximal end and a distal end;
wherein at said proximal end, said waveguide is optically coupled to said radiation source, and said waveguide transmits said radiation to a fistula at its distal end;
a handpiece coupled to said at least one waveguide; and
wherein said radiation source is capable of producing radiation at a preselected wavelength.

9. The device for treating fistulas according to claim 8 wherein said radiation. source is a laser radiation source operating at a wavelength preselected from the group of about 980±30 nm, about 1320±50 nm, about 1470±60 nm, about 2000±50 nm, and combinations of these.

10. The device for treating fistulas according to claim 8 wherein said radiation source operates at a wavelength that is absorbed by a photosensitizer.

11. The device for treating fistulas according to claim 8 wherein said waveguide is a radial emitting optical fiber.

12. The device for treating fistulas according to claim 8 wherein said optical waveguide is hollow along its longitudinal axis.

13. The device for treating fistulas according to claim 12 further comprising means to introduce liquid or viscous substances through said hollow optical fiber.

14. The device for treating fistulas according to claim 7, comprising
at least one radiation source;
a hollow cylindrical handpiece;
at least two optical waveguides, disposed longitudinally on the outer surface of said hollow cylindrical handpiece;
wherein at said proximal end, said waveguides are optically coupled to said radiation source, and said waveguides transmit said radiation to a fistula at their distal end;
wherein said radiation source is capable of producing radiation at a preselected wavelength.

15. The device for treating fistulas according to claim 14 further comprising means to introduce liquid or viscous substances through said hollow handpiece.

16. The device for treating fistulas according to claim 14 wherein said radiation source is a laser radiation source operating at a wavelength preselected from the group of about 980±30 nm, about 1470+60 nm, about 1320±50 nm, about 2000±50 nm, and combinations of these.

17. The device for treating fistulas according to claim 14 wherein said radiation source operates at a wavelength that is absorbed by a photosensitizer.

18. The device for treating fistulas according to claim 14 wherein said waveguide is a radial emitting optical fiber.
